(19) United States
(12) Patent Application Publication
(10) Pub. No.: US 2023/0170054 A1
(43) Pub. Date: Jun. 1, 2023

(54) METHOD OF IDENTIFYING PROPERTIES OF MOLECULES UNDER OPEN BOUNDARY CONDITIONS

(71) Applicant: Purdue Research Foundation, West Lafayette, IN (US)

(72) Inventors: Tillmann Kubis, West Lafayette, IN (US); James Charles, Lafayette, IN (US)

(21) Appl. No.: 18/160,578

(22) Filed: Jan. 27, 2023

Related U.S. Application Data
(63) Continuation-in-part of application No. 18/056,857, filed on Nov. 18, 2022.
Application No. 18/056,857 is a continuation of application No. 16/624,833, filed on Dec. 19, 2019, now Pat. No. 11,508,463.
(60) Provisional application No. 62/526,470, filed on Jun. 29, 2017.

(51) Int. Cl. G16C 10/00; G16C 20/30; G06F 30/28; G06F 17/18

(52) U.S. Cl. CPC G16C 10/00; G16C 20/30; G06F 30/28; G06F 17/18; G06F 2113/08

(57) ABSTRACT

A method of determining a property of a system, the system including at least one molecule in a solvent, comprises: generating a quantum model of the system, the quantum model including a device region and a lead region, the device region being spherical, paraboloid, cubic or arbitrary in shape and encompassing the at least one molecule and a portion of the solvent of the system, the lead region encompassing a region of the solvent surrounding the device region, determining a first property of the device region by solving a first quantum equation for the device region, determining the first property of the lead region by solving the first quantum equation under open boundary conditions for the lead region, and combining the first property of the device region with the first property of the lead region to arrive at a total first property for the system.

### CROSS-REFERENCE TO RELATED APPLICATIONS

This application is a continuation-in-part application of U.S. patent application Ser. No. 18/056,857, filed on Nov. 18, 2022, the disclosure of which is herein incorporated by reference in its entirety. U.S. patent application Ser. No. 18/056,857 is a continuation of U.S. patent application Ser. No. 16/624,833, filed on Dec. 19, 2019, the disclosure of which is herein incorporated by reference in its entirety. U.S. patent application Ser. No. 16/624,833 is a 35 U.S.C. § 371 National Stage Application of PCT/US2018/040348, filed on Jun. 29, 2018, the disclosure of which is herein incorporated by reference in its entirety. PCT/US2018/040348 claims the benefit of priority of U.S. provisional application Ser. No. 62/526,470, filed on Jun. 29, 2017, the disclosure of which is herein incorporated by reference in its entirety.

### TECHNICAL FIELD

The present invention is related to the field of molecular modeling, and, more particularly, to the use of molecular models to identify quantum properties of molecules in liquid systems.

### BACKGROUND

An accurate simulation of the properties and/or behavior of a liquid system, such as a molecule or molecules in a solvent, needs to account for the effects of the bulk medium, or “solvent”, which provides the environment for the molecule of interest. The solvent is typically an aqueous liquid (e.g., water) although it may comprise hydrophobic membranes, other organic or inorganic molecules, emulsions, solids, alloys or mixtures of the above. Important solvent properties include electrostatic screening, cavitation effects, pH, local interactions with other molecules, viscosity, and the provision of a constant-temperature environment. Some or all the solvent's properties may vary spatially. Temporal changes in solvent properties, such as temperature changes, may also occur.

Liquid systems are inherently open quantum systems. In previously known quantum models of open systems, the system is considered as a device connected between two contacts, namely source and drain contacts. The open boundary condition of the system was taken into account by contact self-energies, which represent the charge injection and extraction effect of the contacts. After the contact self-energies are solved, the electronic transport of the system is solved by either non-equilibrium Green's function (NEGF) methods or quantum transmitting boundary method (QTBM) algorithms.

While such previously known methods are effective, the source and drain contacts, which define how the system interacts with the surrounding environment, are finite or semi-finite constructs. In contrast, the contacts/leads of an open system under open boundary conditions are theoretically infinite and extend in all directions. Consequently, the source and drain contacts used in previously known modeling methods do not fully represent an open system under open boundary conditions.

### BRIEF DESCRIPTION OF THE DRAWINGS

FIG. 1 depicts a schematic diagram of an embodiment of a liquid system that is modeled using the modeling system and methods of the present disclosure.

FIG. 2 depicts an embodiment of a model that may be used to model the liquid system of FIG. 1 under open quantum liquid boundary conditions.

FIG. 3 depicts another embodiment of a model that may be used to model the liquid system of FIG. 1 under open quantum liquid boundary conditions.

FIG. 4 depicts yet another embodiment of a model that may be used to model the liquid system of FIG. 1 under open quantum liquid boundary conditions.

FIG. 5 depicts a block diagram of a nanoelectronics modeling system.

FIG. 6 summarizes results of a recursive open boundary and interfaces (ROBIN) method.

FIG. 7 shows a verification of the ROBIN method for electronic material property predictions.

FIG. 8 shows the average DOS of graphene electrons solved in graphene discs of varying diameters.

FIG. 9 shows a periodic distribution of carbon and 3% silicon atoms.

FIG. 10 shows values for graphene with 3% periodically distributed silicon solved with the ROBIN method.

FIG. 11 shows open system results of the center 25 nm of a 200 nm diameter graphene disc with 3% silicon atoms distributed randomly on the left half and periodically on the right half of the disc.

FIG. 12 shows the electronic DOS at the energy of the graphene Dirac point in twisted bilayer graphene with a twist angle of 5° and 30°.

FIG. 13 shows normalized electronic DOS at 50 meV above the valence band edge of Si for a 2 nm spherical Ge cluster embedded in pristine Si solved with the ROBIN method.

FIG. 14 shows the method applied to hollow nanocrystal structures.

FIG. 15 shows the method applied to a solid structure with interfaces and surfaces.

FIG. 16 shows the method applied to carbon nanotube and methanol on graphene.

FIG. 17a-17B show the method applied to two crossing carbon nanotubes.

### DETAILED DESCRIPTION

For the purposes of promoting an understanding of the principles of the disclosure, reference will now be made to the embodiments illustrated in the drawings and described in the following written specification. It is understood that no limitation to the scope of the disclosure is thereby intended. It is further understood that the present disclosure includes any alterations and modifications to the illustrated embodiments and includes further applications of the principles of the disclosure as would normally occur to a person of ordinary skill in the art to which this disclosure pertains.

The present disclosure is directed to methods and systems for modeling a liquid (e.g., molecule/solvent) system that enables the quantum mechanical behavior of the system to be analyzed under open boundary conditions. The model enables open system quantum properties to be calculated for the liquid system. Any kind of observable property may be identified for the liquid system using the model, including density, solubility, reactivity, stability, optical spectra, thermal spectra, magnetic properties, susceptibility, and the like. The model according to the present disclosure is capable of handling any-dimensional open quantum boundary conditions accurately. There is no way currently known to solve open quantum boundaries in three dimensions. All existing methods have only finite-area quantum leads.

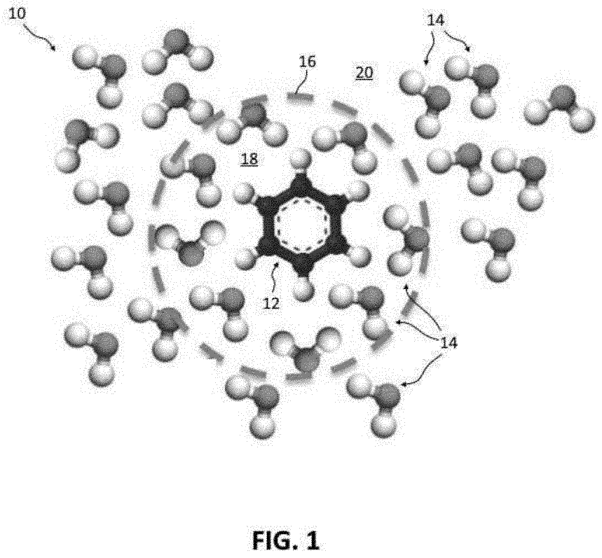

A schematic diagram 10 of a liquid system comprising a molecule 12 in a solvent 14 is depicted in FIG. 1. As used herein, the singular term “molecule” will be used to encompass whatever atomic structure that is to be modeled in the solvent, which can be one or more atoms, one or more molecules, atomic chain(s), solute, and the like. The molecule depicted in the schematic diagram of FIG. 1 is a benzene (C6H6) and the solvent is water. In alternative embodiments, similar models may be generated for substantially any type of molecule in substantially any type of solvent.

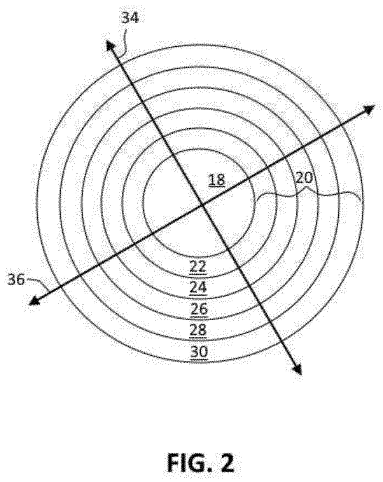

In accordance with the present disclosure, a quantum model of a liquid system, such as the system depicted in FIG. 1, is generated using a suitable simulation/modeling system. An example of such a system is NEMO5. FIG. 2 depicts one embodiment of a quantum model which may be used to model the system of FIG. 1. The liquid system is considered an open system. To model as an open system, the device region is considered as a three-dimensionally shaped region that encompasses the molecule of the liquid system and a portion of the solvent immediately surrounding the molecule.

The model also includes a lead region. As noted above, the lead region was modeled as two contacts, i.e., source and drain contacts, connected to a device in previously known methods which are finite or semi-finite in area and therefore not truly representative of a system under open boundary conditions.

As an alternative to modeling the systems interaction with the surrounding environment using finite or semi-finite leads (e.g., source and drain contacts), the lead region is considered as three-dimensionally shaped region that completely surrounds the device region and has a shape that matches the outer shape of the device region. This configuration for the lead region enables the leads for the device to be handled as being infinite and extending in all directions from the device which is a more accurate representation of the open boundary conditions of an open system, such as a liquid system.

Modeling the liquid system begins with the selection of a base shape for the model which will define the shape of the device region as well as surrounding lead region. Any suitable three-dimensional shape may be used as the base shape for the model. In the embodiment of FIG. 2, the base shape of the model is spherical shape. In alternative embodiments, other shapes may be used, such as cuboid shapes, ovoid shapes, paraboloid shapes, and polyhedrons as well as irregular shapes. Preferably, the base shape of the model is selected to generally follow the shape of the molecule of the liquid system. For example, molecules having an elongated shape can be encompassed by a more elongated three-dimensional as the base shape for the model, such as an ovoid, paraboloid, cylinder, etc.

Dividing the system into a device region and a surrounding lead region, the device region and the lead region can be treated separately in solving quantum equations and determining parameters. The parameter values which are calculated separately for the device region 18 (e.g., P_{d}) and the lead region (P₁) can then be added to arrive at a total value for the parameter (Pₜₒₜₐₗ) for the system (See, e.g., equation (1)).

P_{d}+P₁=Pₜₒₜₐₗ (1)

Partitioning the system into a device region and a lead region enables the system to be analyzed quantum mechanically. One method of analyzing the liquid system model of FIG. 1 is the Non-Equilibrium Green's Function (NEGF) method. The NEGF is the standard approach to model nanoscale open boundary devices, where coherent quantum effects as well as incoherent scattering are present. The NEGF method can be applied to the device region and the lead region separately and then combined to derive properties for the whole system.

The NEGF method requires the solution of the retarded Green's function (G^{R}) and lesser Green's function (G_{<}) in the device to obtain the transmission and the charge density. The key operation of the NEGF method is the inversion of a matrix with the same rank as the device Hamiltonian. However, the solution time and the peak memory usage increases dramatically as the device dimension increases. This is particularly true for spherical leads. For spherical leads, there is a polynomial order 6 relationship between the size, e.g., radius, of the lead and the computation requirements, e.g., inversions, required to analyze the lead quantum mechanically. The computational load (e.g., time and memory) can quickly become intractable with larger radii.

To reduce the computational load of the NEGF method, the recursive Green's function (RGF) may be used. The RGF method is well-known for improving the efficiency of NEGF calculations. It allows solving the transmission and the charge density with only a minimum number of blocks of the G^{R} matrix. The RGF algorithm divides the device into partitions and solves the relevant G^{R} blocks recursively starting with a first partition and continuing in forward direction until a last partition is reached. Afterwards the G^{<} matrix is solved to obtain the charge density.

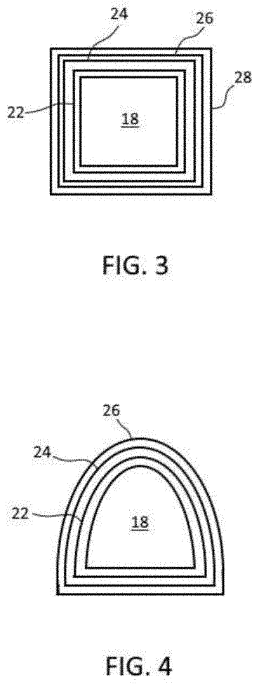

To enable the RGF algorithm to be applied in the present case, the lead region 20 is further divided into a plurality of partitions (or shells) 22, 24, 26, 28, 30. In the embodiment of FIG. 2 having a spherical base shape, the partitions 22, 24, 26, 28, 30 are each spherical shells 22, 24, 26, 28, 30 which are nested inside each other starting at the device/lead interface 16. FIGS. 3 and 4 show the partitioning of the lead region 20 in models having other base shapes. For example, FIG. 3 shows partitioning of the lead region 20 for a model having a device region 18 that is cuboid shaped, and FIG. 4 shows partitioning of the lead region 20 for a model having a device region that is paraboloid shapes.

The surface area and volume of the shells increase with distance from the device region 18. This means that the shell regions which are farther away from the device have more atoms to consider in calculations than the shell regions which are closer to the device.

However, as can be seen in FIG. 2, dephasing increases as the distance from the device region increases (as indicated by arrows 36, 34). A larger dephasing means that the considered physics is more local in real space. This also means that the amount of non-locality decreases with distance from the device region. Because the lead regions farther away from the device region have less non-locality, the amount of time and memory required to perform the calculations in these regions is less in relation to lead regions which are closer to the device region.

Once the value of a particular parameter has been calculated for each of the shell regions of the lead region, the Green's functions of the respective shell regions (g₁₁, g₁₂ . . . g₁ₙ) can then be combined to arrive at the interface Green's function g₁ at the lead/device interface (See, e.g., equation (2)). The device Green's function is then solved with the interface Green's function according to equation (3) and the Keldysh equation. All observables are then deduced from the Green's functions as commonly done in Green's function approaches.

g₁ᵢ=(E−H₁ᵢ−H_{1i,1i-1}g₁ᵢ₋₁H_{1i,1i-1})⁻¹ (2)

G^{R}=(E−H_{d}−H_{d,1}g₁H_{1,d})⁻¹ (3)

Any suitable number of layers and/or thickness of layers may be used in the lead region. In one embodiment, the thickness of the respective shells or partitions depends on the distance range of direct molecule-molecule interactions in the liquid/solvent. With this in mind, the thickness of each shell region layer is preferably kept at a minimum to minimize the computational load for each respective shell region.

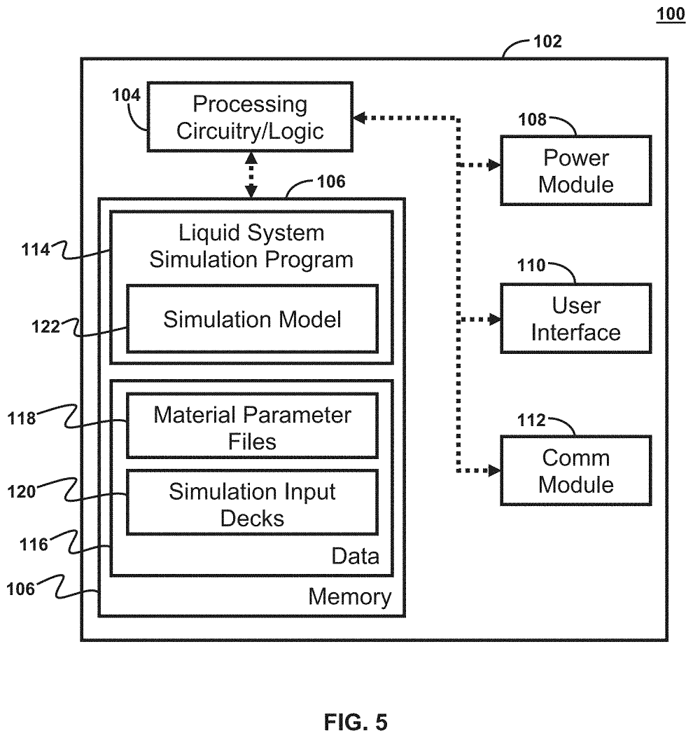

FIG. 5 shows a block diagram of an exemplary embodiment of a liquid system simulation system 100 which can be used to generate, analyze, and/or utilize the model for open quantum liquid boundary conditions discussed above in relation to FIGS. 1 and 2. The liquid system simulation system 100 is typically provided in a housing, cabinet, or the like 102 that is configured in a typical manner for a computing device. In one embodiment, the liquid system simulation system 100 includes processing circuitry/logic 104, memory 106, a power module 108, a user interface 110, and a network communications module 112. It is appreciated that the illustrated embodiment of the liquid system simulation system 100 is only one exemplary embodiment of a liquid system simulation system 100 and is merely representative of any of various manners or configurations of a simulation system, personal computer, server, or any other data processing systems that are operative in the manner set forth herein.

The processing circuitry/logic 104 is configured to execute instructions to operate the liquid system simulation system 100 to enable the features, functionality, characteristics and/or the like as described herein. To this end, the processing circuitry/logic 104 is operably connected to the memory 106, the power module 108, the user interface 110, and the network communications module 112. The processing circuitry/logic 104 generally comprises one or more processors which may operate in parallel or otherwise in concert with one another. It will be recognized by those of ordinary skill in the art that a “processor” includes any hardware system, hardware mechanism or hardware component that processes data, signals, or other information. Accordingly, the processing circuitry/logic 104 may include a system with a central processing unit, multiple processing units, or dedicated circuitry for achieving specific functionality.

The memory 106 may be of any type of device capable of storing information accessible by the processing circuitry/logic 104, such as a memory card, ROM, RAM, write-capable memories, read-only memories, hard drives, discs, flash memory, or any of various other computer-readable medium serving as data storage devices as will be recognized by those of ordinary skill in the art. The memory 106 is configured to store instructions including a liquid system simulation program 114 for execution by the processing circuitry/logic 104, as well as data 116 for use by the liquid system simulation program 114.

With continued reference to FIG. 5, the power module 108 of the liquid system simulation system 100 is configured to supply appropriate electricity to the liquid system simulation system 100 (i.e., including the various components of the liquid system simulation system 100). The power module 108 may operate on standard 120 volt AC electricity, but may alternatively operate on other AC voltages or include DC power supplied by a battery or batteries.

The network communication module 112 of the liquid system simulation system 100 provides an interface that allows for communication with any of various devices using various means. In particular, the network communications module 112 may include a local area network port that allows for communication with any of various local computers housed in the same or nearby facility. In some embodiments, the network communications module 112 further includes a wide area network port that allows for communications with remote computers over the Internet. Alternatively, the liquid system simulation system 100 communicates with the Internet via a separate modem and/or router of the local area network. In one embodiment, the network communications module is equipped with a Wi-Fi transceiver or other wireless communications device. Accordingly, it will be appreciated that communications with the liquid system simulation system 100 may occur via wired communications or via the wireless communications. Communications may be accomplished using any of various known communications protocols.

The liquid system simulation system 100 may be operated locally or remotely by a user. To facilitate local operation, the liquid system simulation system 100 may include an interactive user interface 110. Via the user interface 110, a user may access the instructions, including the liquid system simulation program 114, and may collect data from and store data to the memory 106. In at least one embodiment, the user interface 110 may suitably include an LCD display screen or the like, a mouse or other pointing device, a keyboard or other keypad, speakers, and a microphone, as will be recognized by those of ordinary skill in the art. Alternatively, in some embodiments, a user may operate the liquid system simulation system 100 remotely from another computing device which is in communication therewith via the network communication module 112 and has an analogous user interface.

As discussed above, the liquid system simulation system 100 includes a liquid system simulation program 114 stored in the memory 106. The liquid system simulation program 114 is configured to enable to liquid system simulation system 100 to perform calculations of carrier transport properties, quantum properties and/or other observable characteristics (e.g., density, solubility, reactivity, stability, optical spectra, thermal spectra, magnetic properties, susceptibility, and the like) pertaining to one or more simulation models of the system.

As will be discussed in further detail below, the liquid system simulation program 114 improves upon conventional simulation methods by enabling multi-scale simulations that reflect an accurate and quantitative understanding of quantum mechanics-dominated carrier flow in an entire realistically extended complex device. To accomplish this, the liquid system simulation program 114 partitions a model of a system, such as a liquid system, or molecule in solvent system, into a spherical device region and a plurality of spherical cell lead regions. The simulation program is configured to apply any suitable method or algorithm to the partitioned model to derive selected properties for the system being modeled. Examples of such methods and algorithms include NEGF, RGF, nonlocal RGF, DFT, Wannier Functions, etc.

In one exemplary embodiment, the data 116 includes material parameter files 118 and simulation input decks 120. The material parameter files 118 and simulation input decks 120 include data which defines the structure of the nanoelectronic device to be simulated, as well as various parameters of the simulation to be performed. The material parameter files 118 and/or simulation input decks 120 describe the structure of the liquid system device at an atomic level, and may include information such as geometries, types of materials, doping levels, crystal structures, and other physical characteristics. Additionally, the material parameter files 118 and/or simulation input decks 120 may describe simulation parameters such as bias voltages, input currents, ambient conditions, physical constants, values for experimentally determined parameters, simulation settings, etc. In some embodiments, the simulation input decks 120 are written in a suitable input deck programming language.

The liquid system simulation program 114 receives the material parameter files 118 and simulation input decks 120 as inputs and utilizes one or more models, algorithms, and/or processes to calculate carrier transport characteristics, or other physical phenomena, of the device defined by the respective material parameter files 118 and simulation input decks 120. In at least one embodiment, the liquid system simulation program 114 is configured to provide the calculated carrier transport characteristics or other physical phenomena in the form of an output file which can be used by another program. In some embodiments, the liquid system simulation program 114 is configured to operate a display device of the user interface 110 to display a graphical depiction of the calculated carrier transport characteristics or other physical phenomena, such as a graph, plot, diagram, or the like.

With continued reference to FIG. 5, the liquid system simulation program 114 includes one or more simulation models for open quantum liquid boundary conditions configured to simulate carrier transport characteristics, quantum properties and/or other physical phenomena of a particular liquid system. In the description of the methods and algorithms described herein, statements that the method or model is performing some task or function refers to a general purpose processor, controller, or the like executing programmed instructions stored in non-transitory computer readable storage media operatively connected to the processor to manipulate data or to operate one or more components in the liquid system simulation system 100 to perform the task or function. Particularly, the processing circuitry/logic 104 above may be such a processor and the executed program instructions may be stored in the memory 106. Additionally, the steps of the methods may be performed in any feasible chronological order, regardless of the order shown in the figures or the order in which the steps are described.

### Open Boundary Conditions in Modeling Nonperiodic Materials and Interfaces: The Impact of the Periodicity Assumption

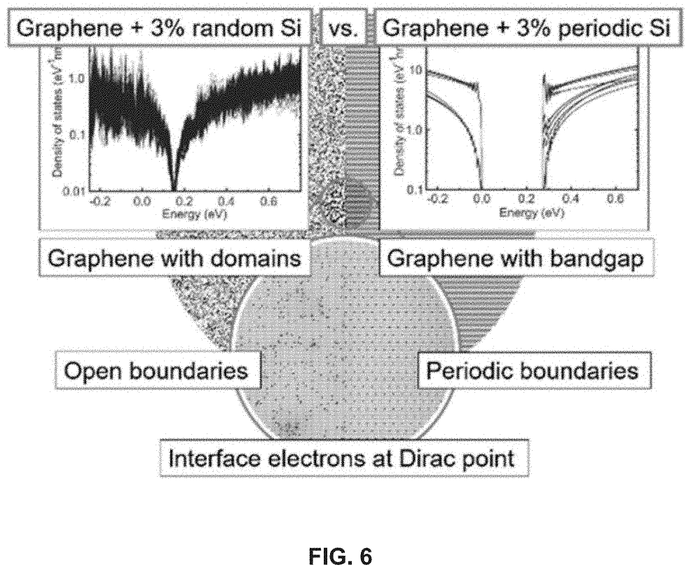

Simulations are essential to accelerate the discovery of new materials and to gain full understanding of known ones. Although hard to realize experimentally, periodic boundary conditions are omnipresent in material simulations. In this description, we introduce ROBIN (recursive open boundary and interfaces), the first method allowing open boundary conditions in material and interface modeling. The computational costs are limited to solving quantum properties in a focus area that allows explicitly discretizing millions of atoms in real space and to consider virtually any type of environment (be it periodic, regular, or random). The impact of the periodicity assumption is assessed in detail with silicon dopants in graphene. As summarized in FIG. 6, graphene was confirmed to produce a band gap with periodic substitution of 3% carbon with silicon in agreement with published periodic boundary condition calculations. Instead, 3% randomly distributed silicon in graphene only shifts the energy spectrum. The predicted shift agrees quantitatively with published experimental data. Key insight of this assessment is, assuming periodicity elevates a small perturbation of a periodic cell into a strong impact on the material property prediction. Periodic boundary conditions can be applied on truly periodic systems only. More general systems should apply an open boundary method for reliable predictions.

Computer-aided material predictions represent the first-step of many new material discoveries. Material simulations can power machine learning searches for new materials with specific properties. However, modeling experimental reality with wide-spread idealized, periodic boundary conditions is prone to artifacts: Irregular interfaces, impurities, cracks and dislocations are not compatible with idealized conditions. A common approach to limit artificial periodicity effects is to make the repeating unit cell as large as numerically feasible and apply various correction algorithms.

Instead, we introduce the recursive open boundary and interfaces (ROBIN) method, which can handle arbitrary geometries and atom distributions and does not need any periodicity assumption. It is based on the nonequilibrium Green's function method (NEGF). The NEGF method had been applied on charge, spin, and heat transport in open nanodevices. The ROBIN extension of NEGF models materials in infinitely extended real space and supports regular and irregular systems. We verify the ROBIN method in 2D and 3D crystalline systems. To assess the impact of periodic boundary conditions on material property predictions, an as-simple-as-possible but experimentally realized system was chosen: Calculations of graphene confirm recent work that periodically distributed silicon impurities can open bandgaps. In stark contrast and presumably closer to any experiment, random distributions of the same amount of silicon are shown to give no band gaps but to form domains and to linearly shift the band structure. The predicted shift quantitatively agrees with experimental data of the publication “Opening the Band Gap of Graphene through Silicon Doping for the Improved Performance of Graphene/GaAs Heterojunction Solar Cells” (Zhang, S. J.; Lin, S. S.; Li, X. Q.; Liu, X. Y.; Wu, H. A.; Xu, W. L.; Wang, P.; Wu, Z. Q.; Zhong, H. K.; Xu, Z. J. Nanoscale 2016, 8, 226-232). The findings of ROBIN are analyzed in detail and show that periodic boundary conditions can elevate otherwise small perturbations to systematic changes of material properties.

So far, all models for quantum electronic material properties are based on Hermitian Hamiltonian operators (H) that represent either periodic or finite-sized systems. The boundaries of closed systems yield confinement effects and system size dependent resonances that can interfere with the actual material properties. Models with periodic boundary conditions require numerically hard to achieve unit cell sizes to avoid artificial long-distance coupling between repeating simulation domain features. To lift some of the numerical limitations of periodic simulations, various correction methods have been introduced. The k-space sampling required for periodic boundary simulations represents additional numerical challenges. Modeling systems with long distance effects, such as Moiré lattices, systems with irregularities, such as alloys, and systems with inhomogeneous fields or strain are notoriously difficult to handle with Hermitian Hamiltonian operators.

In the NEGF method, the electronic density of states (DOS) equals the imaginary part of the retarded Green's function's (G^{R}) diagonal. G^{R} is solved in the Dyson equation, which reads in operator form G^{R}=(E−H_{c}−Σ^{R})−1, with the electronic energy E, and the retarded self-energy Σ^{R}. The Hermitian Hamiltonian HC represents the electrons in the finite, central area C. We set C to be a sphere for three-dimensional and a circle for two-dimensional systems. However, any other space-appropriate shapes are possible, too. Electrons are modeled in the effective mass approximation when the ROBIN method is verified against analytical DOS of parabolic dispersions in 2D and 3D. In case of graphene, electrons are given in single-orbital atomistic tight binding (E_{PzC}=9, V_{PPσ,C}=0, V_{PPπ}=−3 eV), following the nomenclature of the publication “Compact Expression for the Angular Dependence of Tight-Binding Hamiltonian Matrix Elements” (Podolskiy, A. V.; Vogl, P. Phys. Rev. B: Condens. Matter Mater. Phys. 2004, 69, 233101) on the native graphene lattice. Silicon atoms in graphene are modeled with graphene parameters and an on-site energy of E_{Pz}, Si=4.75 eV to reproduce the band gap of 3% periodically distributed Si in graphene predicted with DFT in the publication “Opening the Band Gap of Graphene through Silicon Doping for the Improved Performance of Graphene/GaAs Heterojunction Solar Cells.” Note that many other electronic representations, such as plane waves, maximally localized Wannier functions, or localized atomic orbitals, have been applied in NEGF before. Devices modeled in NEGF covered 1D, 2D, and 3D symmetries, ranging from molecular junctions up to micrometer long resistors.

The retarded self-energy E^{R} is the key element that distinguishes NEGF from closed-system models: It is the non-Hermitian operator in the inverse G^{R} that represents the interaction of electrons in C with the surrounding of C at the contact interface between the two regions. Σ^{R} allows electrons to enter and leave C at the contact and then to propagate to infinite distance to C. The imaginary part of E^{R} is inverse proportional to the electronic lifetime in C (i.e., the “dwelling-in-C-time”).

Most NEGF applications require the surrounding “behind” the contact to form a homogeneous lead and in particular to have a well-defined 1D transport direction. A few exceptions to this limitation can be found for quantum cascade systems and recent transistor predictions. The publication “Non-Equilibrium Green's Functions Method: Non-Trivial and Disordered Leads” (He, Y.; Wang, Y.; Klimeck, G.; Kubis, T. Appl. Phys. Lett. 2014, 105, 213502), in particular, allowed for the lead cross section size to grow infinitely with increasing distance to the contact and to host random atom distributions.

The ROBIN method expands the contact self-energy method of the publication “Non-Equilibrium Green's Functions Method: Non-Trivial and Disordered Leads” by considering the total interface between C and the surrounding as the contact area. The conceptual difference to the publication “Non-Equilibrium Green's Functions Method: Non-Trivial and Disordered Leads” is the fact that only one contact self-energy describes the complete environment. Following the publication “Non-Equilibrium Green's Functions Method: Non-Trivial and Disordered Leads”, the non-Hermitian Σ^{R} is solved as a product of the non-Hermitian surface retarded Green's function of the 2D or 3D surrounding of C with the Hermitian Hamiltonian operators of atoms in C coupling with atoms in the surrounding. Thereby, the environment atoms are discretized explicitly. A complex absorbing potential (CAP) is added to the environmental atoms' on-site energies. Similar to that in the publication “Non-Equilibrium Green's Functions Method: Non-Trivial and Disordered Leads,” the CAP vanishes at the edges of C and grows smoothly with increasing distance to C. The CAP is critical to ensure efficient convergence of the results in C with the range of explicitly discretized surrounding atoms.

The numerical costs solving for the retarded Green's functions is the largest challenge of the ROBIN method. Therefore, all retarded Green's functions are solved recursively to limit the required peak memory and to allow for explicit consideration of up to 3 million atoms in this description. Many publications and online lectures on recursive Green's functions describe the method in high detail. Details of the CAP method are discussed in the publication “Non-Equilibrium Green's Functions Method: Non-Trivial and Disordered Leads” and the publication “General Retarded Contact Self-Energies in and beyond the Non-Equilibrium Green's Functions Method” (Kubis, T.; He, Y.; Andrawis, R.; Klimeck, G. J. Phys.: Conf. Ser. 2016, 696, No. 012019). All ROBIN calculations have been performed on 10 nodes of the Brown cluster of the Rosen Center for Advanced Computing at Purdue University.

Since all density of states results of open system calculations come with a continuous DOS, smoothing spectral results as needed in Hermitian models is obsolete here. Although this description covers only electronic examples, the presented method applies to any system with discretizable equations of motion, including, for example, lattice vibrations in dynamic matrix descriptions.

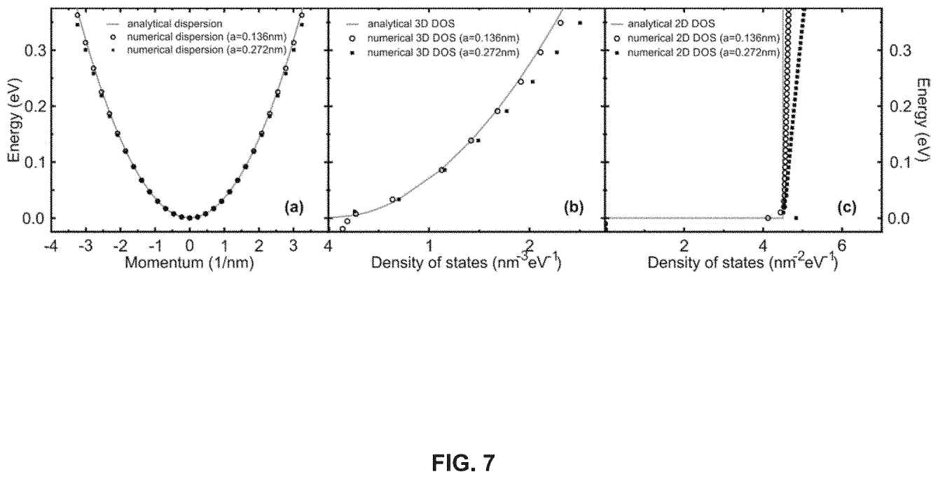

FIG. 7 verifies the ROBIN method for electronic material property predictions. FIG. 7a is a reminder of the electronic dispersion resulting of electronic Hamiltonian operators of silicon conduction band electrons (m*=1.08 m₀) discretized in real space and solved with periodic boundary conditions. Note this is the only periodic-boundary system result, while all remaining results apply the ROBIN method of open boundaries. Deviations from the analytical parabolic dispersion become smaller with decreasing kinetic energies and finer mesh spacing. Accordingly, with finer real space meshes and smaller kinetic energies the DOS of the ROBIN method in 3D (FIGS. 7b) and 2D (FIG. 7c) agree better with the respective analytical DOS, that is, the square root function in 3D and the constant DOS in 2D.

In FIG. 7: Verification of the ROBIN method against analytical results: (a) the analytical dispersion of effective mass electrons (line) and the numerical dispersion of periodic Hamiltonian operators discretized in a real space mesh of 0.136 (circles) and 0.272 nm (dots) mesh point distance are known to deviate more with higher kinetic energies. Similarly, the numerical density of states of open system simulations with ROBIN (symbols) deviates from the analytical one (lines) with higher energies and larger mesh constants, both in 3D (b) and 2D (c). Otherwise, all results of the ROBIN method resemble the expected analytical data very well.

Similar to the Si nanowire calculations in the publication “Opening the Band Gap of Graphene through Silicon Doping for the Improved Performance of Graphene/GaAs Heterojunction Solar Cells,” the convergence of Σ^{R} close to band edges is more demanding and small deviations from the analytical DOS can be observed there. Better convergence further reduces the DOS deviation at the band edge.

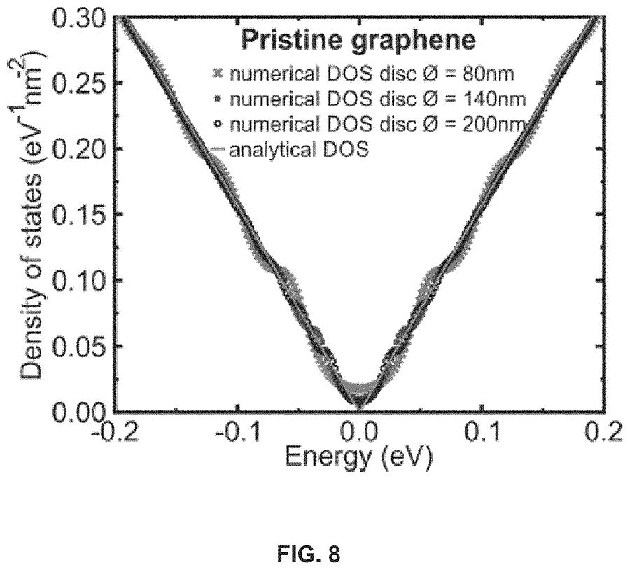

This convergence also determines the quality of the predicted DOS at the Dirac point of graphene. FIG. 8 shows the average DOS of graphene electrons solved in graphene discs of varying diameters. The center region C is chosen to be a disc of 1 nm diameter for all results in FIG. 8.

In FIG. 8: Verification of the ROBIN method against analytical results: The numerical density of states resulting of the ROBIN method (symbols) of graphene discs agree better with the analytical density of states (line) with larger discretized disc diameter.

All remaining carbon atoms are included as part of the environment of C within the ROBIN method. In this way, the largest disc size considered in FIG. 8 is 200 nm diameter, which includes more than 1 million discretized carbon atoms in total. The average DOS in FIG. 8 converges well to the linear dispersion of graphene with increasing lead size. Simultaneously, the standard deviation of the DOS of each considered atom in C versus the depicted average value reduces, too. The maximum of this standard deviation for all considered energies in FIG. 8 is 1.2 ×10−4 (80 nm), 1.5×10−5 (140 nm), and 6.3×10−7 eV−1 nm−2 (200 nm), respectively.

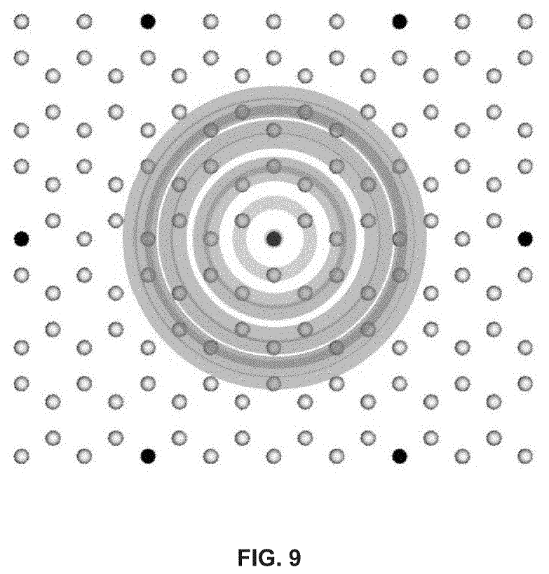

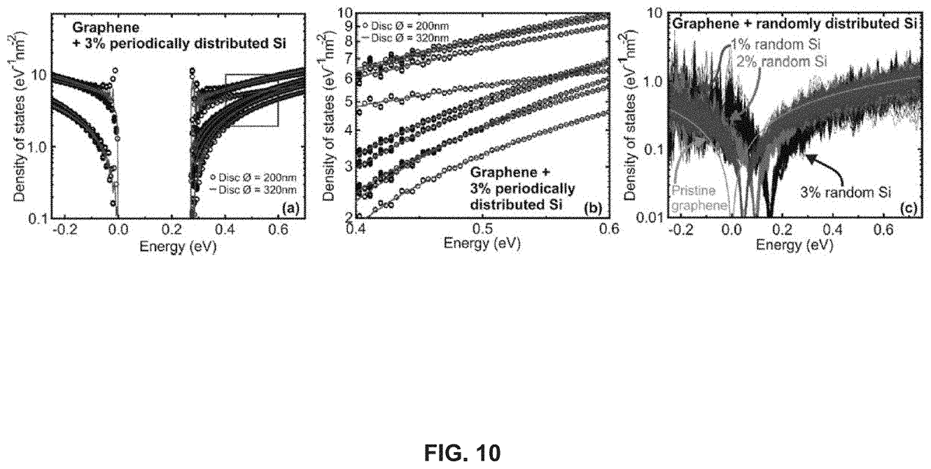

In the publication “Opening the Band Gap of Graphene through Silicon Doping for the Improved Performance of Graphene/GaAs Heterojunction Solar Cells,” a 3% concentration of periodically distributed silicon atoms in graphene was analyzed with density functional theory calculations and periodic boundary conditions. It was predicted that the addition of the silicon atoms opens a bandgap of 0.28 eV in graphene. This finding can be reproduced with the ROBIN method in empirical tight binding: All Si atoms are considered periodically distributed in the graphene disc. Silicon parameters are approximated with graphene parameters and an additional on-site energy of 4.75 eV. Given the unit cell is larger with the periodic Si than in the case of pristine graphene (see FIG. 9), the convergence of the DOS with respect to the disc diameter is numerically more challenging. This can be seen in the slowly decaying beating pattern in FIG. 10a. Even a disc diameter of 320 nm with more than 3 million discretized atoms still shows a small beating in the resulting DOS around the band gap. FIG. 10a shows the electronic DOS of each of 282 atoms of a 3 nm center area of two different graphene discs (200 and 320 nm diameter) solved with the ROBIN method.

The periodic distribution of carbon (white) and 3% silicon (black) atoms is shown in FIG. 9. The addition of silicon atoms increases the graphene unit cell to 32 atoms that fall into 9 different chemical categories: 1 silicon atom and 8 graphene atoms in 8 different distances to the silicon one (see FIG. 9). Accordingly, a ROBIN prediction of the atom resolved DOS of graphene with 3% periodically distributed Si yields 9 different DOS lines, as shown in FIG. 10a. Note that FIG. 10a actually shows 282 individual DOS lines for each of the 282 atoms in the 3 nm center region. Good convergence of the contact self-energy makes them virtually identical to DOS lines of atoms with the same chemical environment (see FIG. 10b for a zoom-in).

In FIG. 9: Schematic of carbon (white) and silicon (black) atoms in graphene with 3% periodically distributed silicon. Nine different atom types are in each unit cell: one silicon atom and carbon atoms in 8 different distances to the central silicon, highlighted by 8 semi-transparent rings around the center Si circle.

The DOS changes significantly when the 3% silicon atoms are randomly distributed (see FIG. 10c). The 282 local DOS lines of each atom in the center region C differ depending on their respective local atomic environment. The ensemble of atomic DOS lines maintains a Dirac point at about ΔE=0.147 eV above the Dirac point of pristine graphene. Note that AF scales approximately linearly with the % fraction of randomly distributed Si atoms in graphene as can be seen in FIG. 10c for the 1% and 2% Si cases. For comparison, FIG. 10c also shows the analytical DOS of pristine graphene.

Adding only 1%, 2%, or 3% silicon should only perturb graphene within the linear response regime. Indeed, the ROBIN results in FIG. 10c for this amount of randomly distributed Si show only a linear shift of the Dirac cone. Periodic boundary conditions of the same small amount of Si atoms give a dramatic change to the graphene band structure, resembling effectively a new material. In other words, applying periodic boundary conditions elevates otherwise small perturbations to systematic material property changes. Therefore, periodic boundary conditions should only be applied to truly periodic systems. In the experiments of the publication “Opening the Band Gap of Graphene through Silicon Doping for the Improved Performance of Graphene/GaAs Heterojunction Solar Cells,” 3% randomly distributed Si in graphene yielded a shift of the electronic work function by 0.13 eV. The predicted shift of 147 meV in FIG. 10c agrees quantitatively with that observation given the experimental Si concentration uncertainty of the publication “Opening the Band Gap of Graphene through Silicon Doping for the Improved Performance of Graphene/GaAs Heterojunction Solar Cells.” (2.7-4.5%).

In FIG. 10, (a) Density of states of graphene with 3% periodically distributed silicon solved with the ROBIN method reproduces the 0.28 eV band gap of the publication “Opening the Band Gap of Graphene through Silicon Doping for the Improved Performance of Graphene/GaAs Heterojunction Solar Cells,” when the on-site energy of Si is chosen as 4.75 eV. The large unit cell of 3% Si in graphene burdens the numerical convergence w.r.t the disc diameter. 200 nm (symbols) and, to lesser extent, the 320 nm (lines) disc diameter show incomplete convergence near the band gap. (b) Zoom-in into the boxed region in panel a. The 282 individual atoms of the calculation in panel a fall into 9 distinct groups of DOS lines, corresponding to the 9 different atom types shown in FIG. 9. (c) The DOS solved in the ROBIN method of randomly distributed Si atoms in graphene does not show a bandgap. Instead, increasing Si content shifts the DOS to higher energies by about 47 meV per Si-percentage (i.e., about 1% of the assumed on-site energy difference of carbon and silicon atoms). The line shows the analytical DOS of pristine graphene for comparison.

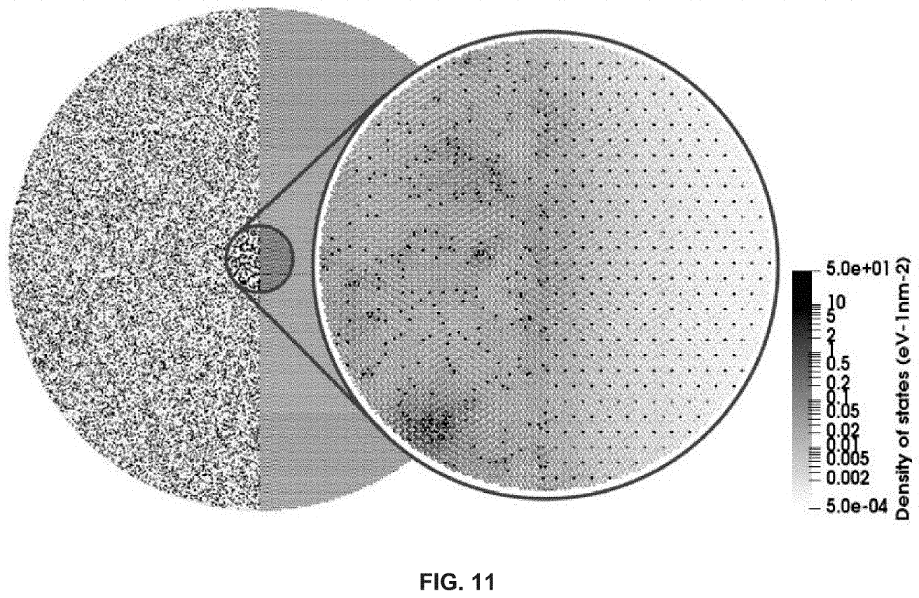

To illustrate the DOS difference of periodically and randomly distributed silicon atoms in graphene, FIG. 11 shows open system results of the center 25 nm of a 200 nm diameter graphene disc with 3% silicon atoms distributed randomly on the left half and periodically on the right half of the disc. The contour shows the position resolved DOS at the energy of 10 meV above the Dirac point of pristine graphene. The black spheres indicate the position of Si atoms. Depending on local Si atom distributions, electrons on the left face pockets of high DOS; whereas, all the DOS decays in the right are due to the bandgap opened by the periodically distributed Si.

In FIG. 11. (left) 200 nm disc of graphene (carbon atoms are white) with 3% Si atoms (black) distributed randomly on the left and periodically on the right half of the disc. (right) Electronic density of states of the center 25 nm of the 200 nm graphene disc solved with open boundary conditions at 10 meV above the Dirac point of pristine graphene. Carbon atoms are shaded according to the electronic DOS, and the silicon atoms are black. The electronic DOS shows domain formation in the left half and electronic tunneling into the right half of the disc.

Substituting atoms periodically is a remarkably difficult experimental task especially if single substitutions are considered. We expect random distributions to resemble the experimental reality much more closely. Given the stark contrast in electronic properties of periodic versus random distributions, materials with periodic substitutions should be considered fully distinct from the original pristine host material. This applies to substituting with other than Si atom kinds, as well as other host materials than graphene.

In conclusion, this description introduces the ROBIN method to predict 2D and 3D materials in arbitrary, regular, and irregular atomic compositions. Green's functions are solved recursively to explicitly discretize millions of atoms within the memory limitations of typical state of the art hardware. When applied on silicon atoms distributed in graphene, the method reveals a significant difference in the electronic properties of periodic versus randomly distributed Si atoms in graphene. The calculations confirm periodically distributed Si atoms form bandgaps in graphene, but the same amount of randomly distributed Si atoms forms domains in the electronic DOS and shifts the graphene DOS in energy. The results show that applying periodic boundary conditions can elevate small perturbations to massively influence material property predictions.

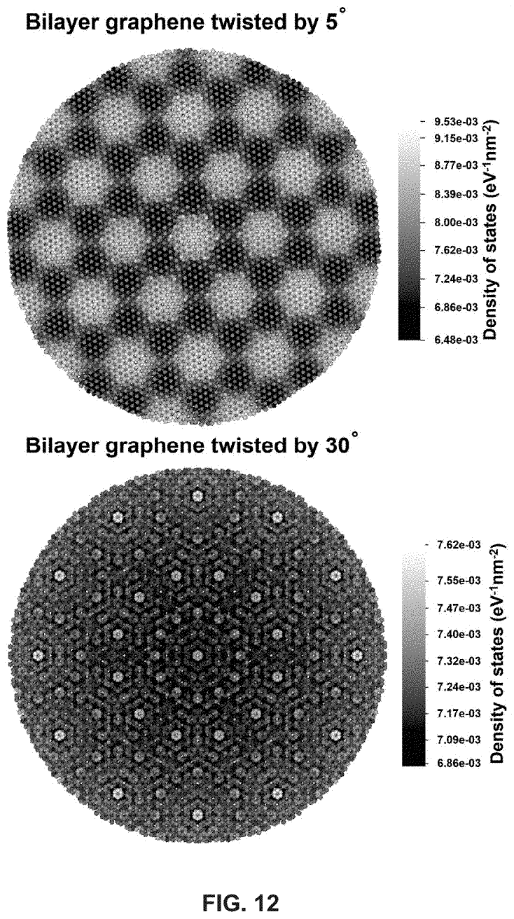

It is worth mentioning that the ROBIN method can be applied on systems with random alloys, single defects, and interfaces. Systems involving different physical phases (e.g. heterogeneous catalysis, emulsions, melting solids, microdroplet chemistry, etc.) are conceptually equivalent to the situation in FIG. 11. To illustrate the generality of the ROBIN method, FIG. 12 shows the electronic DOS at the energy of the graphene Dirac point in twisted bilayer graphene with a twist angle of 5° (a) and 30° (b), respectively. The ROBIN method is independent of whether the system is irregular, periodic, or quasi-crystalline. The data in the FIG. 12 were solved with the same numerical effort and the same simulation settings. The expected periodicity and quasi-crystalline behavior is reproduced in both cases.

In FIG. 12, Electronic DOS at the energy of the graphene Dirac point in twisted bilayer graphene with a twist angle of 5 degrees (a) and 30 degrees (b) solved with the ROBIN method. Bilayer graphene parameters of NEMO5's database were altered with Harrison scaling for the electronic Hamiltonian. The results of both angles were solved with the same number of explicitly discretized atoms, independent of the resulting DOS distribution.

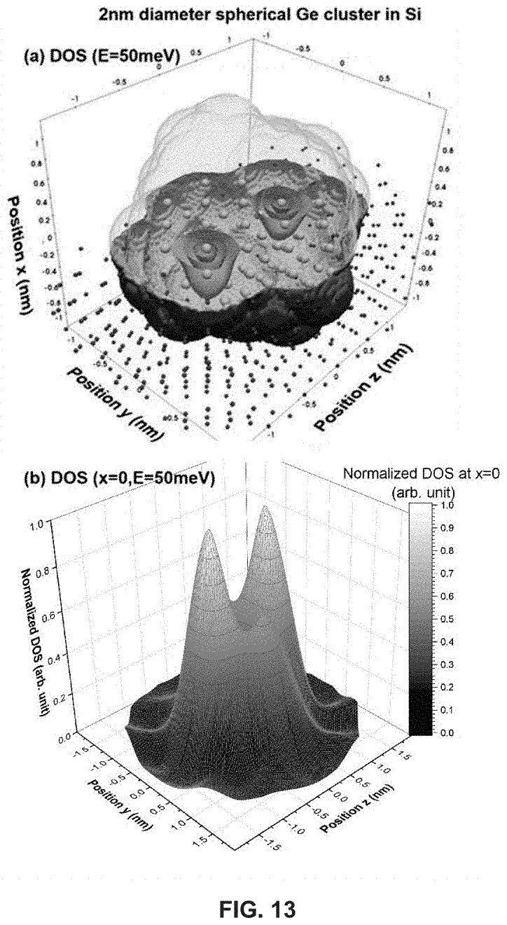

The applicability of ROBIN on 3D materials is further exemplified in FIG. 13 because it shows the electronic DOS of a spherical Ge cluster embedded in Si and solved with ROBIN in sp3d5s* atomistic tight binding representation. The cluster size is chosen to be 2 nm in diameter, which is common in SiGe alloys. The electronic DOS at 50 meV above the Si valence band edge has two maxima in the Ge cluster but leaks significantly into Si.

In FIG. 13, Normalized electronic DOS at 50 meV above the valence band edge of Si for a 2 nm spherical Ge cluster embedded in pristine Si solved with the ROBIN method. For better visibility, Si (purple) and Ge atoms (yellow) only up to x=0 are shown (a). The contour graph (a) and the cut of the DOS at x=0 (b) show the electronic DOS leaks significantly into the Si domain.

### Applications of the Methods to Solid Structures

As discussed above, in addition to aqueous liquids, a solvent may comprise hydrophobic membranes, other organic or inorganic molecules, emulsions, solids, alloys or mixtures of the above. Accordingly, it should be appreciated that the methods discussed above are applicable to a wide variety of bulk mediums that incorporate one or more molecules of interest arranged therein.

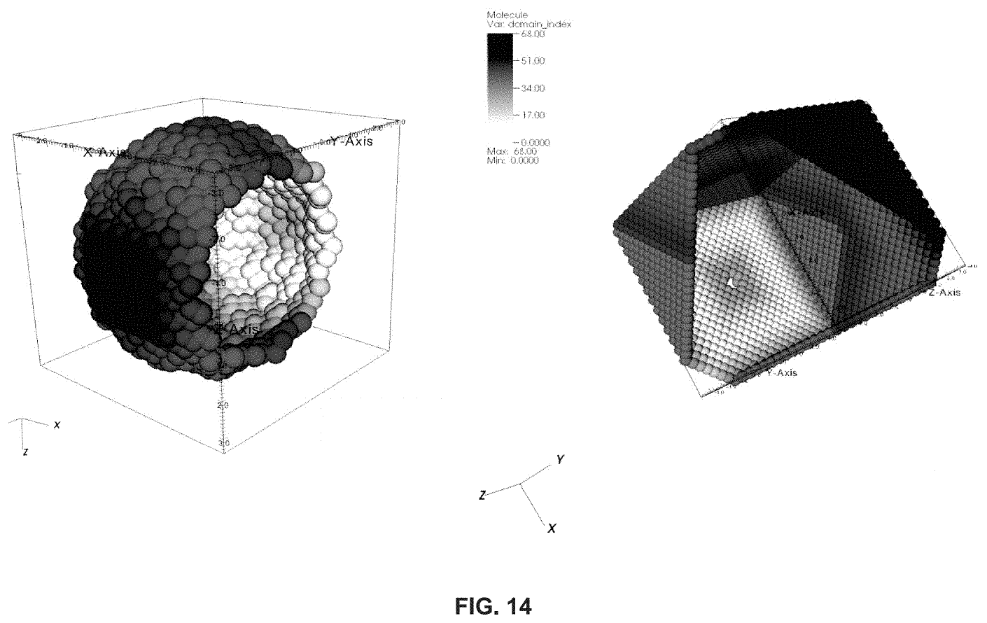

FIG. 14 shows two exemplary nanocrystal structures to which the methods discussed above can be applied. As can be seen, the nanocrystal structures each include a hollow region. Although not shown, each of the exemplary nanocrystal structures incorporates at least one molecule of interest for simulation. A device region is defined encompassing the at least one molecule, and a plurality of nested shell regions are defined extending outward through the respective nanocrystal structure. In the illustration, the device region, within which the at least one molecule of interest is located, is shaded with a darkest shading (i.e., black). Additionally, each of the nested shell regions is shaded with different shades of grey to identify each nested shell region. Each nested shell region can be understood as individual subgroup of the atoms in the nanocrystal structures. In this way, much like in the examples discussed above, setting up the respective quantum model for the each of the nanocrystal structures is a matter of categorizing the atoms of the nanocrystal structures into subgroups. (e.g., device region, first shell region, second shell region, third shell region, etc.).

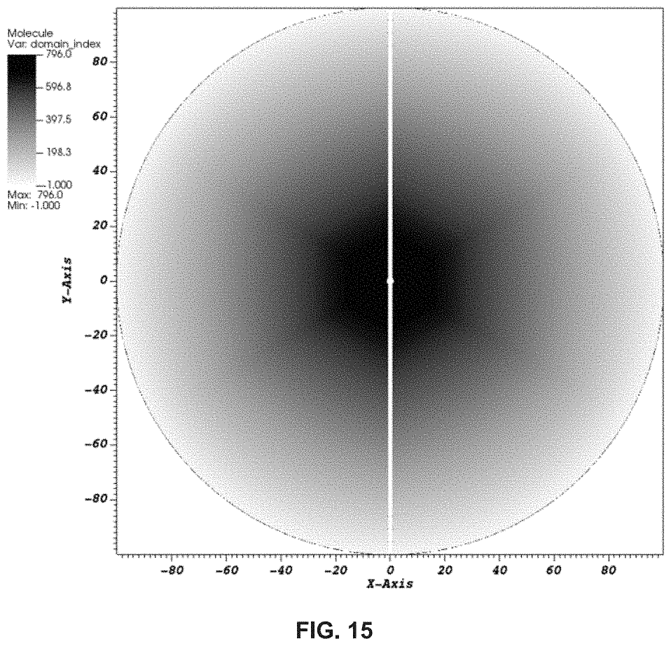

FIG. 15 shows a further exemplary structure to which the methods discussed above can be applied, in which the structure has interfaces and surfaces. In particular, the exemplary structure is a graphene disc with a slit. Although not shown, at least one molecule of interest for simulation is incorporated near the center of the graphene disc. As before, a device region, which is shaded with the darkest shading, is defined encompassing the at least one molecule. Likewise, a plurality of nested shell regions, which are shaded with different shades of grey, are defined extending outward through the graphene disc. This example of a graphene disc with a slit illustrates that the subgroups of atoms with a structure (e.g., device region, first shell region, second shell region, third shell region, etc.) may be defined discontinuously.

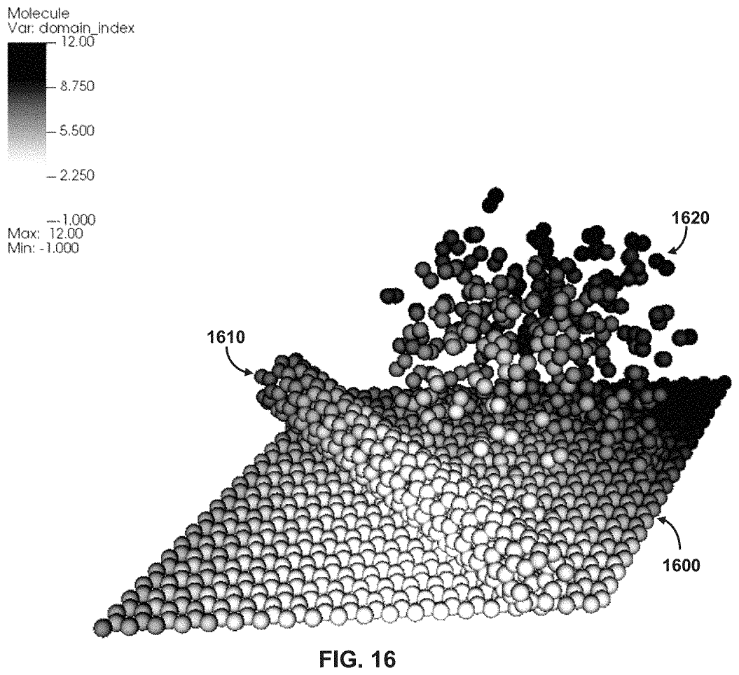

FIG. 16 shows an exemplary set of structures to which the methods discussed above can be applied, in which the structure incorporates a solids-solid interface, a solid-liquid interface, a solid-gas interface, a gas-liquid interface, and molecules adsorbed onto a solid surface. In particular, the exemplary set of structures includes a graphene sheet 1600, with a carbon nanotube 1610 situated in contact with the graphene sheet 1600 and liquid methanol 1620 situated nearby and at least partially in contact with the graphene sheet 1600. Although not shown, at least one molecule of interest for simulation is incorporated in the set of structures. As before, a device region, which is shaded with the darkest shading, is defined encompassing the at least one molecule. Likewise, a plurality of nested shell regions, which are shaded with different shades of grey, are defined extending outward through the graphene disc. In this example, the set of structures can be split into two subdomains: an interior subdomain and an exterior subdomain. The interior subdomain can be solved as a total domain. The exterior subdomain can be split into subgroups, as before (e.g., device region, first shell region, second shell region, third shell region, etc.).

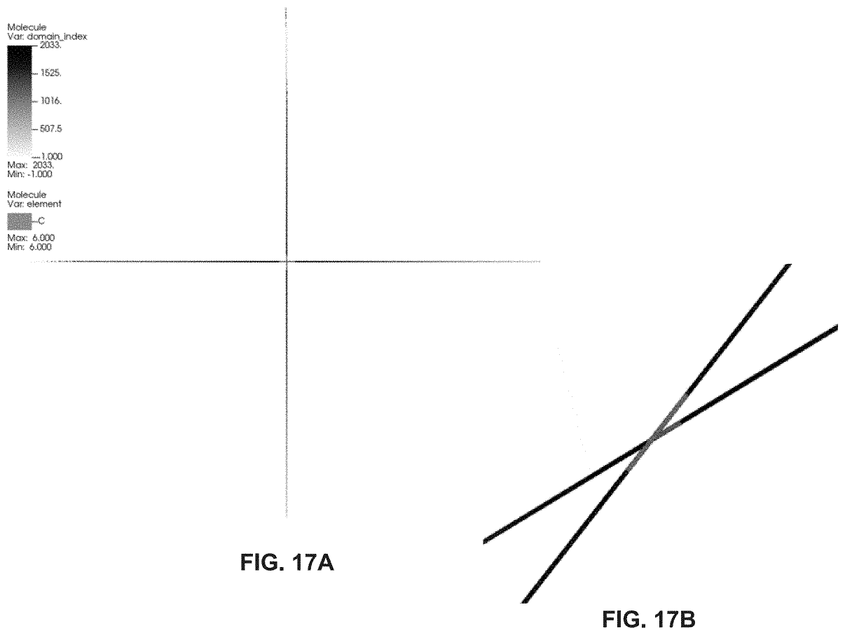

FIGS. 17A and 17B shows a pair of carbon crossing nanotubes from different viewing angles. Although not shown, at least one molecule of interest for simulation is incorporated into the pair of carbon nanotubes near their crossing. As before, a device region, which is shaded with the darkest shading, is defined encompassing the at least one molecule. Likewise, a plurality of nested shell regions, which are shaded with different shades of grey, are defined extending outward through the graphene disc. In this example, the set of structures can be split into two subdomains: an interior subdomain and an exterior subdomain. The interior subdomain can be solved as a total domain. The exterior subdomain can be split into subgroups, as before (e.g., device region, first shell region, second shell region, third shell region, etc.). Accordingly, in this way, the methods can be applied to irregular solids and carbon nanotube networks.

While the disclosure has been illustrated and described in detail in the drawings and foregoing description, the same should be considered as illustrative and not restrictive in character. It is understood that only the preferred embodiments have been presented and that all changes, modifications and further applications that come within the spirit of the disclosure are desired to be protected.

What is claimed is:

1. A method for simulating a nanoscale device using a modeling system, the nanoscale device including a system having at least one molecule in a solvent, the method comprising:
receiving model parameters for the system as input to a processor of the modeling system, the model parameters identifying at least one of a type of molecule and a type of solvent to be modeled for the system;
generating a quantum model of the system using the processor, the quantum model partitioning the system into a device region and a lead region, the device region encompassing the at least one molecule and a portion of the solvent of the system, the lead region being further partitioned into a plurality of nested shell regions, each nested shell region encompassing a respective region of the solvent surrounding the device region in the system, the plurality of nested shell regions being arranged in a nested manner starting from a device-lead interface and extending outward from the device region, the device-lead interface defining where the device region meets the lead region; and
simulating the nanoscale device using the processor based on the quantum model, the simulating including:
determining a first property of the lead region using Non-Equilibrium Green's Function methods under open boundary conditions for the lead region using the processor, a recursive Green's function algorithm being applied to the plurality of nested shell regions to determine Green's functions for the plurality of nested shell regions of the lead region;
determining the first property of the device region using Non-Equilibrium Green's Function methods using the processor, a Green's function for the device region being determined based on a Green's function for the device-lead interface, the Green's function for the device-lead interface being determined based on the Green's functions for the plurality of nested shell regions of the lead region; and
combining the first property of the device region with the first property of the lead region to arrive at a total first property for the system using the processor.

2. The method of claim 1, further comprising:
determining a Hamiltonian for the system; and
determining the Green's function for the device with reference to the Hamiltonian.

3. The method of claim 2, wherein the Hamiltonian is determined using a Wannierization procedure.

4. The method of claim 1, wherein dephasing increases with distance from the device region which results in the nested shell regions that are farther away from the device region having less non-locality than the nested shell regions that are closer to the device region, and
wherein a number of matrix inversions required to solve Green's functions for a given region depends in part on the amount of non-locality in the region.

5. The method of claim 1, wherein the solvent comprises at least one of hydrophobic membranes, organic molecules, inorganic molecules, emulsions, solids, and alloys.

6. The method of claim 1, wherein the solvent comprises as crystal structure that incorporates the at least one molecule.

7. The method of claim 1, wherein the solvent comprises as solid bulk medium that incorporates the at least one molecule.

8. The method of claim 7, wherein the solid bulk medium includes at least one of a graphene disc and a carbon nanotube.

9. A method for simulating a nanoscale device using a modeling system, the nanoscale device including a system having at least one molecule incorporated in a solid bulk medium, the method comprising:
receiving model parameters for the system as input to a processor of the modeling system, the model parameters identifying at least one of a type of molecule and a type of solid bulk medium to be modeled for the system;
generating a quantum model of the system using the processor, the quantum model partitioning the system into a device region and a lead region, the device region encompassing the at least one molecule and a portion of the solid bulk medium of the system, the lead region being further partitioned into a plurality of nested shell regions, each nested shell region encompassing a respective region of the solid bulk medium surrounding the device region in the system, the plurality of nested shell regions being arranged in a nested manner starting from a device-lead interface and extending outward from the device region, the device-lead interface defining where the device region meets the lead region; and
simulating the nanoscale device using the processor based on the quantum model, the simulating including:
determining a first property of the lead region using Non-Equilibrium Green's Function methods under open boundary conditions for the lead region using the processor, a recursive Green's function algorithm being applied to the plurality of nested shell regions to determine Green's functions for the plurality of nested shell regions of the lead region;
determining the first property of the device region using Non-Equilibrium Green's Function methods using the processor, a Green's function for the device region being determined based on a Green's function for the device-lead interface, the Green's function for the device-lead interface being determined based on the Green's functions for the plurality of nested shell regions of the lead region; and
combining the first property of the device region with the first property of the lead region to arrive at a total first property for the system using the processor.

10. The method of claim 9, further comprising:
determining a Hamiltonian for the system; and
determining the Green's function for the device with reference to the Hamiltonian.

11. The method of claim 10, wherein the Hamiltonian is determined using a Wannierization procedure.

12. The method of claim 9, wherein dephasing increases with distance from the device region which results in the nested shell regions that are farther away from the device region having less non-locality than the nested shell regions that are closer to the device region, and
wherein a number of matrix inversions required to solve Green's functions for a given region depends in part on the amount of non-locality in the region.

13. The method of claim 9, wherein the solid bulk medium comprises as crystal structure that incorporates the at least one molecule.

14. The method of claim 9, wherein the solid bulk medium includes at least one of a graphene disc and a carbon nanotube.

15. A method for simulating a nanoscale device using a modeling system, the nanoscale device including a system having at least one molecule incorporated in a medium, the method comprising:
receiving model parameters for the system as input to a processor of the modeling system, the model parameters identifying at least one of a type of molecule and a type of medium to be modeled for the system;
generating a quantum model of the system using the processor, the quantum model partitioning the system into a device region and a lead region, the device region encompassing the at least one molecule and a portion of the medium of the system, the lead region being further partitioned into a plurality of nested shell regions, each nested shell region encompassing a respective region of the medium surrounding the device region in the system, the plurality of nested shell regions being arranged in a nested manner starting from a device-lead interface and extending outward from the device region, the device-lead interface defining where the device region meets the lead region; and
simulating the nanoscale device using the processor based on the quantum model, the simulating including:
determining a first property of the lead region using Non-Equilibrium Green's Function methods under open boundary conditions for the lead region using the processor, a recursive Green's function algorithm being applied to the plurality of nested shell regions to determine Green's functions for the plurality of nested shell regions of the lead region;
determining the first property of the device region using Non-Equilibrium Green's Function methods using the processor, a Green's function for the device region being determined based on a Green's function for the device-lead interface, the Green's function for the device-lead interface being determined based on the Green's functions for the plurality of nested shell regions of the lead region; and
combining the first property of the device region with the first property of the lead region to arrive at a total first property for the system using the processor.

16. The method of claim 15, further comprising:
determining a Hamiltonian for the system; and
determining the Green's function for the device with reference to the Hamiltonian.

17. The method of claim 16, wherein the Hamiltonian is determined using a Wannierization procedure.

18. The method of claim 15, wherein dephasing increases with distance from the device region which results in the nested shell regions that are farther away from the device region having less non-locality than the nested shell regions that are closer to the device region, and
wherein a number of matrix inversions required to solve Green's functions for a given region depends in part on the amount of non-locality in the region.

19. The method of claim 15, wherein the medium comprises as crystal structure that incorporates the at least one molecule.

20. The method of claim 15, wherein the medium includes at least one of a graphene disc and a carbon nanotube.
